(19) United States
(12) Patent Application Publication
(10) Pub. No.: US 2016/0202228 A1
(43) Pub. Date: Jul. 14, 2016

(54) WATER QUALITY MONITORING AND EARLY EVENT DETECTION

(71) Applicant: International Business Machines Corporation, Armonk, NY (US)

(72) Inventors: AMADOU BA, DUBLIN (IE); SEAN MCKENNA, DUBLIN (IE)

(21) Appl. No.: 14/591,949

(22) Filed: Jan. 8, 2015

(51) Int. Cl. G01N 33/18; G08B 21/18

(52) U.S. Cl. CPC G01N 33/18; G01N 33/182; G08B 21/182

(57) ABSTRACT

According to one exemplary embodiment, a method for water quality monitoring and early detection of a contamination event is provided. The method can include receiving a first water characteristic value corresponding with a water characteristic. The method can include determining a second water characteristic value corresponding with the water characteristic using a prediction algorithm having a tunable parameter, based on the first water characteristic value and the tunable parameter. The method can include comparing the first water characteristic value and the second water characteristic value. The method can include determining a residual value, based on the comparing. The method can include updating the tunable parameter, based on the determined residual value. The method can include determining if the contamination event is present, based on the residual value. The method can include sending an alert, based on determining the presence of the contamination event.

### BACKGROUND

The present invention relates generally to the field of computing, and more particularly to water quality monitoring.

Growing world population and attendant depletion of water resources require increasing exploitation of hitherto untapped water resources. Utilizing untapped water resources such as desalinized seawater, saline groundwater, or water from rivers have become more important to meet global demand. The process of transforming non-drinkable water to drinkable water can require a monitoring procedure that can be an assessment of the conformity of transformed water to specified objectives. Traditional methods for water quality monitoring can collect water samples that can then be preserved, transported, and analyzed at a laboratory.

### SUMMARY

According to one exemplary embodiment, a method for water quality monitoring and early detection of a contamination event by a recorder device is provided. The method can include receiving a first water characteristic value corresponding with a water characteristic. The method can also include determining a second water characteristic value corresponding with the water characteristic using a prediction algorithm having a tunable parameter, based on the first water characteristic value and the tunable parameter. The method can then include comparing the first water characteristic value and the second water characteristic value. The method can further include determining a residual value, based on comparing the first water characteristic value to the second water characteristic value. The method can include updating the tunable parameter, based on the determined residual value. The method can also include determining if the contamination event is present, based on the residual value. The method can then include sending an alert, based on determining the presence of the contamination event.

According to another exemplary embodiment, a computer system for water quality monitoring and early detection of a contamination event by a recorder device is provided. The computer system can include one or more processors, one or more computer-readable memories, one or more computer-readable tangible storage devices, and program instructions stored on at least one of the one or more storage devices for execution by at least one of the one or more processors via at least one of the one or more memories, whereby the computer system is capable of performing a method. The method can include receiving a first water characteristic value corresponding with a water characteristic. The method can also include determining a second water characteristic value corresponding with the water characteristic using a prediction algorithm having a tunable parameter, based on the first water characteristic value and the tunable parameter. The method can then include comparing the first water characteristic value and the second water characteristic value. The method can further include determining a residual value, based on comparing the first water characteristic value to the second water characteristic value. The method can include updating the tunable parameter, based on the determined residual value. The method can also include determining if the contamination event is present, based on the residual value. The method can then include sending an alert, based on determining the presence of the contamination event.

According to yet another exemplary embodiment, a computer program product for water quality monitoring and early detection of a contamination event by a recorder device is provided. The computer program product can include one or more computer-readable storage devices and program instructions stored on at least one of the one or more tangible storage devices, the program instructions executable by a processor. The computer program product can include program instructions to receive a first water characteristic value corresponding with a water characteristic. The computer program product can also include program instructions to determine a second water characteristic value corresponding with the water characteristic using a prediction algorithm having a tunable parameter, based on the first water characteristic value and the tunable parameter. The computer program product can then include program instructions to compare the first water characteristic value and the second water characteristic value. The computer program product can further include program instructions to determine a residual value, based on comparing the first water characteristic value to the second water characteristic value. The computer program product can include program instructions to update the tunable parameter, based on the determined residual value. The computer program product can also include program instructions to determine if the contamination event is present, based on the residual value. The computer program product can then include program instructions to send an alert, based on determining the presence of the contamination event.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flow chart illustrating a process for a water quality monitoring according to at least one embodiment; and

FIG. 3 is a block diagram of internal and external components of computers and servers depicted in FIG. 1 according to at least one embodiment.

### DETAILED DESCRIPTION

Detailed embodiments of the claimed structures and methods are disclosed herein; however, it can be understood that the disclosed embodiments are merely illustrative of the claimed structures and methods that can be embodied in various forms. This invention can, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques can be omitted to avoid unnecessarily obscuring the presented embodiments.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the block can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for water quality monitoring. Additionally, the present embodiment has the capacity to improve the technical field of water quality monitoring by incorporating a mobile water testing device and utilizing algorithms that detect water quality anomalies earlier.

As described previously, given the essential nature of water for human survival and the growth of human populations worldwide, tapping otherwise unusable water sources has become imperative. Desalinizing salt water or treating other water to be safe for human consumption provides a way for more water to be available to meet demand. Processing non-drinkable water into drinkable water typically can be done in high volume in order to economically provide enough drinkable water to support a large population. Transforming non-drinkable water into drinkable water can also require consistent water quality monitoring to ensure that the water is uniformly being processed into drinkable water. Traditional models of water quality monitoring can include taking water samples, transporting the samples to a laboratory, and then analyzing the samples at the laboratory. Traditional models can encounter two major problems. First, the extent to which the water sample can be representative of the water source of interest, as many water sources vary with time and location. Second, once the sample can be removed from the water source, the water source can begin to establish a new equilibrium with the water source's surroundings that can lead to discrepancies between variables and actual conditions. Furthermore, the time between sample collections can restrict real-time water quality monitoring. As a result, a pollution event or a containment event can occur before detection can prevent serious damage.

Therefore, it would be advantageous to, among other things, provide a way to sample water quality in real-time, without removing sample water from the water source, and rapid initialization prediction methods to quickly predict water quality and detect anomalies.

According to at least one embodiment, a dual device system can be utilized that can include a reader device and a recorder device for water quality monitoring. The recorder device can record water quality characteristics (i.e., pH, turbidity, chlorine, temperature, etc.) in time series (i.e., water quality sampled at regular time intervals), perform water quality predictions, and compute residuals. If the recorder device predicts a water quality abnormality, the recorder device can send an alert to the reader device and the reader device can then relay the alert to service personnel. The recorder device can include a mobile robot with water quality sampling capabilities (e.g., a self-propelled submersible robot) or by fixed sensors. The reader device can include an electronic device, such as a computer or server that can be deactivated until an alert from the recorder device is sent to the reader device.

According to at least one embodiment, the recorder device can be initialized and begin water quality data acquisition after initialization. Water quality data can be collected via sensors attached to the fixed or mobile recorder device. The accuracy of water quality measurements can then be determined. If water quality measurements can not be correct, the recorder device can be reinitialized, data can be reacquired, and accuracy can be determined again. If the water quality measurements repeatedly fail to meet accuracy thresholds, an alert can be generated. However, if water quality readings are determined to be accurate, a data cleaning process can be performed on the acquired water quality time series data. Data cleaning can eliminate outlier readings and compensate for missing water characteristic values (e.g., pH level) detected in the acquired data by, for example, using a median filter.

The recording device can then generate water quality predictions based on the cleaned data. The prediction can estimate the next water quality value (e.g., pH level) of the water quality time series. To predict water quality values, various models can be employed. According to at least one embodiment, a black box model can be used. It can be appreciated that other models can be used for prediction. For example, a first-order auto-regressive model can perform the prediction. A first-order auto-regressive model can be built by considering that the output of the model can be obtained from a first-order lagged input. The prediction can be performed by identifying first, at each time step, the auto-regressive parameter, which in turn can allow the output (i.e., the predicted water quality value) to be predicted at a given time step. Due to the complex characteristics of water quality data (e.g., non-stationary data collection and non-linear data), the prediction process can be achieved with a kernel recursive least squares algorithm where the main tuning parameter can be the kernel bandwidth.

During data prediction, the difference between the measured value and the previously predicted value can be determined and a residual value can be generated representing the difference. In the absence of abnormal events, the determined residual value can be zero and the process can continue forming predictions of water quality without change to the tunable parameters of the prediction algorithm. Conversely, in the presence of abnormal events (e.g., a contamination event), the determined residual value can result in a nonzero number indicating the predicted values and measured values can not match. A nonzero residual value can have a sign and magnitude that can indicate how the measured water value and predicted water value differ. The prediction algorithm can also use adaptive tuning parameters to generate predictions. In the absence of anomalies, the tuning parameters can be fixed. However, in the presence of a contamination event, the tuning parameters can become variable in order to improve prediction accuracy and determined residual accuracy. In the case of a nonzero residual value, new tuning parameters can be estimated based on the residual value to accelerate convergence and allow faster anomaly detection.

Determined residual values can then be used to detect anomalies. According to at least one embodiment, statistical hypothesis testing can be used for detecting anomalies based on determined residuals. The generated residuals can be considered random variables, where the residual value's mean and variance in absence of contamination events can be used for contamination event detection. According to at least one other embodiment, online Bayesian change-point detection can be used to detect anomalies. Using online Bayesian change-point detection, the probability of the occurrence of a contamination event can be determined based on the measured water quality values.

If the recorder device detects a contamination event, the reader device (e.g., computer, server, etc.) can be notified by the recorder device of the exact location, the abnormal water characteristic, and the nature and severity of the anomaly.

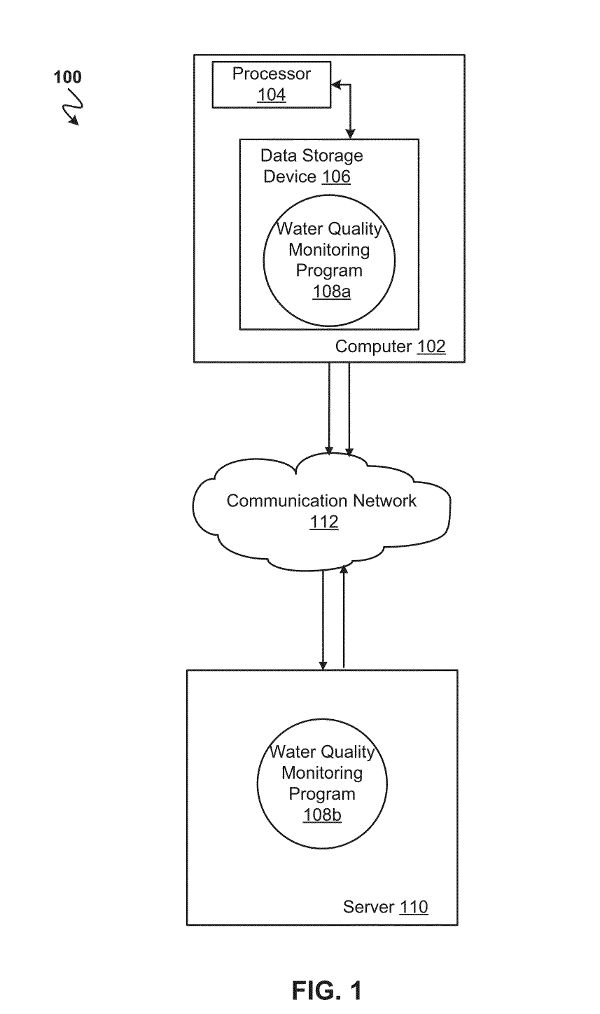

Referring now to FIG. 1, an exemplary networked computer environment 100 in accordance with one embodiment is depicted. The networked computer environment 100 can include a computer 102 with a processor 104 and a data storage device 106 that is enabled to run a water quality monitoring program 108a. The networked computer environment 100 can also include a server 110 that is enabled to run a water quality monitoring program 108b and a communication network 112. The networked computer environment 100 can include a plurality of computers 102 and servers 110, only one of which is shown for illustrative brevity. The communication network can include various types of communication networks, such as a wide area network (WAN), local area network (LAN), a telecommunication network, a wireless network, a public switched network and/or a satellite network. It can be appreciated that FIG. 1 provides only an illustration of one implementation and does not imply any limitations with regard to the environments in which different embodiments can be implemented. Many modifications to the depicted environments can be made based on design and implementation requirements.

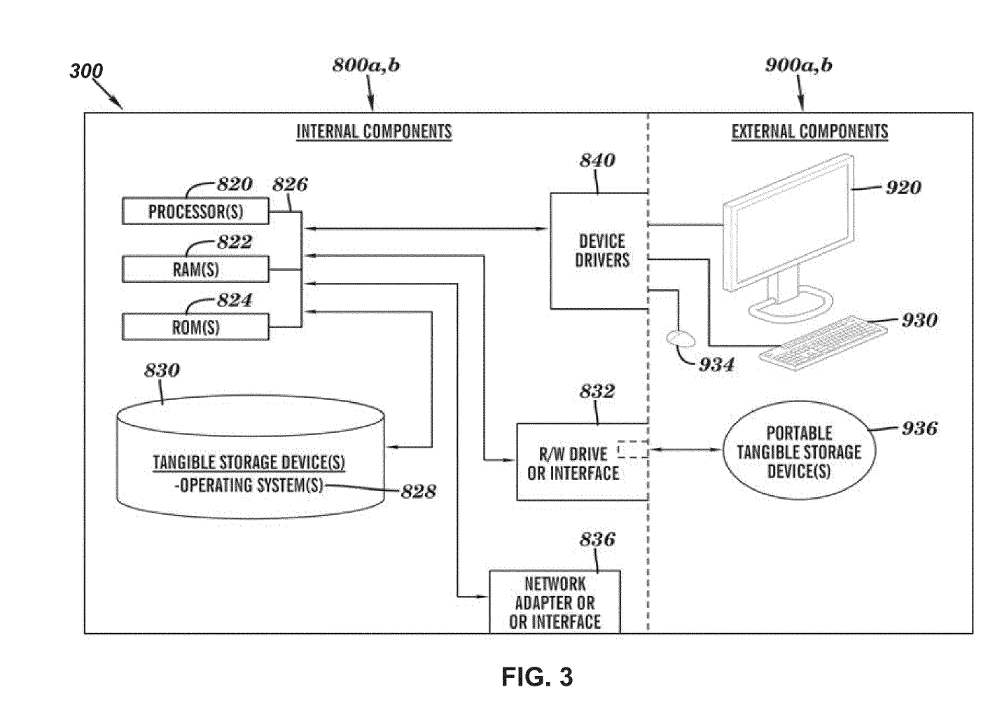

The client computer 102 can communicate with server computer 110 via the communications network 112. The communications network 112 can include connections, such as wire, wireless communication links, or fiber optic cables. As will be discussed with reference to FIG. 3, server computer 110 can include internal components 800a and external components 900a, respectively and client computer 102 can include internal components 800b and external components 900b, respectively. Client computer 102 can be, for example, a mobile device, a telephone, a PDA, a netbook, a laptop computer, a tablet computer, a desktop computer, or any type of computing device capable of running a program and accessing a network.

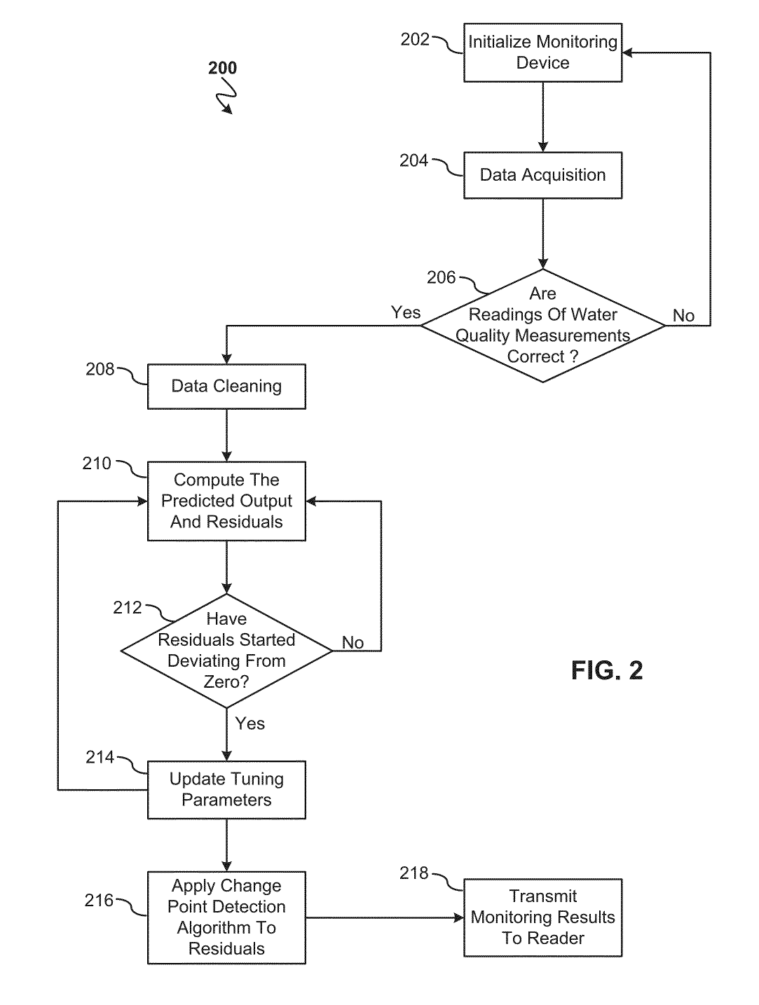

A program, such as a water quality monitoring program 108a and 108b can run on the client computer 102 or on the server computer 110. The water quality monitoring program 108a and 108b can run on a recording device to assess water quality and detect anomalies that can then be forwarded to a reader device. The water quality monitoring program 108a and 108b is explained in further detail below with respect to FIG. 2.

Referring now to FIG. 2, an operational flow chart illustrating the exemplary process 200 by the water quality monitoring program 108a and 108b (FIG. 1) according to at least one embodiment is depicted.

At 202, the recording device can be initialized. According to at least one embodiment, the process 200 can be initialized and run on the recording device. The recording device can be a mobile or fixed water quality sampling station that can sample water quality in real-time. For example, a mobile water quality sampling station can be implemented as a self-propelled submersible robot having attached sensors to read water characteristics (e.g., pH, chlorine, turbidity, temperature, etc.). The submersible robot can traverse a water source and dynamically sample water quality at different locations within the water source. Multiple fixed water quality sampling stations can also be employed having sensors submerged in a water source. Initialization can include booting a computer (e.g., 102 (FIG. 1)) within the recording device and starting the process 200. Additionally, recording device hardware systems can start (e.g., sensors, propulsion and navigation systems, etc.).

Next, at 204, water quality data can be acquired by the recording device. According to at least one embodiment, sensors accessible by the process 200 can sample water at the sensors and generate water quality data (e.g., pH level, chlorine level, etc.) based on the sensors' readings. Water quality data, including a timestamp, can be saved in a data repository, such as a database accessible by the process 200.

Then, at 206, the process 200 can determine if the water quality measurements acquired at 204 are correct. According to at least one embodiment, the process 200 can determine that the measurements are incorrect if data can be missing. For example, if the water quality measurement corresponding to a single time step within a time series can be missing a water characteristic reading, such as chlorine, the process 200 can determine that the measurements can be incorrect and reinitialize the recording device.

If the process 200 determines that the water quality measurements can not be correct at 206, the process 200 can discard the incorrect data and return to 202 to reinitialize the recording device. According to at least one embodiment, a counter can be used to record the number of times that water quality readings can be incorrect and can generate a warning if the number of incorrect readings exceeds a predetermined threshold (e.g., five consecutive times, or ten times overall, etc.) that can indicate a hardware issue with the recording device.

However, if the process 200 determines that the water quality readings can be correct, the process 200 can perform data cleaning on the acquired water quality data at 208. According to at least one embodiment, the process 200 can clean acquired data by compensating for missing data and/or identifying and eliminating outlier data. Data cleaning can be implemented through the use of a known algorithm, such as median filtering. Median filtering can be used to fill in data values (e.g., chlorine level value) missing from water quality data steps with a median value. The median of the value can be determined from the last recorded value before the missing value and the first recorded value after the missing value. It can be appreciated that other filters or methods can be employed to predict or otherwise compensate for missing values.

At 210, the process 200 can compute the predicted output and residuals. According to at least one embodiment, the process 200 can utilize the acquired water quality data to predict what the water quality data can be in the next time step (i.e., when the next water data sample can be collected) within the time series with a prediction algorithm. According to at least one implementation, a known black-box model can be employed, such as a first-order autoregressive model to perform prediction using another known algorithm, such as a kernel recursive least squares algorithm to generate filter coefficients for the black-box model. An autoregressive model specifies that future values can be estimated based on a weighted sum of past values. The first order autoregressive model can be built by considering that the output of the model can be obtained from the first order lagged input parameter. According to at least one implementation, an autoregressive model can be expressed as:

$${y}_{t} = {\sum }_{i = 1}^{l} {a}_{i} {y}_{t - 1} + {ɛ}_{t} ,$$

where {aᵢ}ᵢ₌₁^{l} can be fixed coefficients and Et can be noise with a mean of zero and variance of σ². Water quality output yₜ can be used to determine the water quality input xₜ, that can be the lagged input yₜ₋ₜ, where t can be the time instant and i=1 to l can be the order of the autoregressive model. In order to reach the predicted water quality yₜ, the coefficients (i.e., {aᵢ}ᵢ₌₁^{l}) of the autoregressive model can be computed first.

According to at least one implementation, the coefficients of the black-box model (e.g., autoregressive model) can be computed using a known algorithm, such as a kernel recursive least squares algorithm. The kernel recursive least squares algorithm can be used to recursively find a filter coefficient that minimizes a weighted linear least squares cost function relating to the input parameter.

Water quality, as a parameter in the kernel recursive least squares algorithm, can be any recorded measurement describing water quality such as chlorine, turbidity, pH , etc. that can be represented by yₜ. Considering N water quality samples {(xₜ, yₜ)}Nₜ₌₁^{N}, xₜ can be the lagged yₜ. Determining the autoregressive algorithm coefficients relating yₜ to xₜ can amount to learning an underlying function. Any solution to the problem can be represented in the form f(·)=Σₜ₌₁^{N}αₜK(χₜ, ·), where α can be the parameters to be determined. The resulting optimization of the problem can be

$${min}_{α ∈ {R}^{N}} {‖ y - Φ α ‖}^{2} + {γα}^{T} Φα , \qquad ( 1 )$$

where y=[y₁, . . . , y_{N}] is the N×1 vector of water quality measurement, Φ=[₁(x₁), . . . , _{N}(X_{N})] is the N×N kernel matrix of input vectors, which can be the lagged water quality measurement, [Φ]ᵢⱼ=K(xᵢ, xⱼ) which can be the kernel function in the work that can use a Gaussian kernel as the kernel function and α is a N×1 parameter vector. With the matrix inversion lemma the parameters a can be determined by solving

α=Φ(γI+Φ^{T}Φ)⁻¹y. (2)

The above equation can require α matrix inversion, which can be difficult to achieve for high-dimensional data. Therefore, the parameters a can be recursively determined with kernel recursive least squares.

To estimate recursively α, the time evolution of the equation (2) can be written as

αₜ=Φₜ(γI+Φₜ^{T}Φₜ)⁻¹yₜ. (3)

The kernel method allows the computation of Φₜ^{T}Φₜ. Furthermore, the matrix inversion lemma allows the determination of Kₜ=(γI+Φₜ^{T}Φₜ)⁻¹. By denoting the linear combination of the transformed data by αₜ=Φₜωₜ, where ωₜ are the expansion coefficients and κₜ=Φₜ₋₁^{T}ₜ, the resulting equations can be

$${K}_{t} = {ψ}_{t}^{- 1} [ \begin{matrix}{K}_{t - 1} {ψ}_{t} + {θ}_{t} {θ}_{t}^{T} & - {θ}_{t} \\ - {θ}_{t}^{T} & 1\end{matrix} ] , \qquad ( 4 )$$

ωₜ=[ωₜ₋₁−θₜΨₜ⁻¹εₜ, Ψₜ⁻¹εₜ]^{T}, (5)

where θₜ=Kₜ₋₁κₜ, Ψₜ=γ+ₜ^{T}ₜ−θₜ^{T}κₜ, and εₜ=yₜ−κₜ^{T}ωₜ₋₁ is the prediction error, determined by the difference between the desired signal and the prediction, which can also be called residual values. A summary of kernel recursive least squares will be described below with reference to the kernel recursive least squares algorithm. In the kernel recursive least squares algorithm, the kernel bandwidth can be fixed σ and can play a factor in the accuracy of the prediction and consequently in the residual εₜ. A low kernel bandwidth value can generate high fluctuations in the predicted water quality ŷₜ=κₜ^{T}ωₜ₋₁ (that can lead to false alarms during the monitoring process) and a high kernel bandwidth value can reduce the prediction accuracy (that can lead to delay in detection during the monitoring process). To establish a trade-off along the kernel bandwidth size range, the kernel bandwidth can be determined online.

For example, a kernel recursive least squares algorithm can have as input K₁=(γ+k(x₁, x₁))⁻¹, kernel bandwidth σ, regularization γ, ω₁=K₁y₁, for t=2,3, . . .

First, the Gaussian kernel function can be computed,

$$k ( x , {x}^{′} ) = {e}^{\frac{- {( x - {x}^{′} )}^{2}}{2 {σ}^{2}}} . \qquad$$

Form the Gaussian kernel vector,

κₜ=[k(xₜ, x₁), . . . ,k(xₜ, xₜ₋₁)]^{T}.

Weight the Gaussian kernel vector with the Gram matrix Kₜ₋₁,

θₜ=Kₜ₋₁κₜ.

Compute the kernel function at the actual point χₜ,

$$k ( {x}_{t} , {x}_{t} ) = \begin{matrix}T & \\ t & t .\end{matrix}$$

Compute the scaling Ψₜ,

$${ψ}_{t} = γ + \begin{matrix}T & \\ t & t\end{matrix} - {θ}_{t}^{T} {κ}_{t} .$$

Compute the gram matrix Kₜ,

$${K}_{t} = {ψ}_{t}^{- 1} [ \begin{matrix}{K}_{t - 1} {ψ}_{t} + {θ}_{t} {θ}_{t}^{T} & - {θ}_{t} \\ - {θ}_{t}^{T} & 1\end{matrix} ] .$$

Compute the residuals εₜ,

εₜ=yₜ−κₜ^{T}ωₜ₋₁.

Finally, update the expansion coefficients ωₜ,

$${ω}_{t} = [ \begin{matrix}{ω}_{t - 1} - {θ}_{t} {ψ}_{t}^{- 1} {ɛ}_{t} \\ {ψ}_{t}^{- 1} {ɛ}_{t}\end{matrix} ] .$$

According to at least one implementation, a gradient descent method with a momentum term can be used to determine kernel bandwidth online. Therefore, the updating of the kernel bandwidth can be done with the equation:

σₜ=σₜ₋₁−η∇_{σ}_{ₜ₋₁}(f_{σ}_{ₜ})+μ(σₜ₋₁−σₜ₋₂), (6)

where, σₜ is the kernel bandwidth of the Gaussian kernel function, f_{σ}_{ₜ}=1/2E[εₜ²] is the cost function, η>0 is the learning rate and μ>0 is the momentum rate. The above algorithm incorporates the incremental quantity μ(σₜ₋₁−σₜ₋₂) to the gradient descent method, where μ is a small positive scalar denoting momentum rate. The additional term corresponds to the previous change in the kernel bandwidth. If the change in the previous kernel bandwidth is large, then adding a fraction of the quantity to the current update can accelerate the descent process towards the global minimum and result in the faster convergence. The residual value can be described as εₜ=yₜ−κₜ^{Tω}ₜ₋₁. Then

∇_{σ}_{ₜ₋₁}(f_{σ}_{ₜ})=E[εₜ]∇_{σ}_{ₜ₋₁}(E[εₜ]).

The expectation symbol can be dropped as the expectation can be taken at every time step. The computation of ∇_{σ}_{ₜ₋₁}(εₜ) results in

∇_{σ}_{ₜ₋₁}(εₜ)=−└∇_{σ}_{ₜ₋₁}(κₜ^{T})ωₜ₋₁+κₜ^{T}∇_{σ}_{ₜ₋₁}(ωₜ₋₁)┘,

where the gradient of the kernel function can be determined from the gradient of the current and all the previous input vectors

∇_{σ}_{ₜ₋₁}(κₜ)=└∇_{σ}_{ₜ₋₁}k(xₜ, x₁), . . . , ∇_{σ}_{ₜ₋₁}k(xₜ, xₜ₋₁)┘,

The gradient of the expansion coefficients can be determined with respect to σₜ₋₁, ∇_{σ}_{ₜ₋₁}(ωₜ), resulting in

∇_{σ}_{ₜ₋₁}(ωₜ)=[ω̅1ₜω̅2ₜ]^{T},

where

ω̅1ₜ=∇_{σ}_{ₜ₋₁}(ωₜ₋₁)=└∇_{σ}_{ₜ₋₁}(θₜ)Ψₜ⁻¹εₜ

+θₜ∇_{σ}_{ₜ₋₁}(Ψₜ⁻¹)εₜ+θₜΨₜ⁻¹∇_{σ}_{ₜ₋₁}(εₜ)┘,

and

ω2ₜ=∇_{σ}_{ₜ₋₁}(Ψₜ⁻¹)εₜ+Ψₜ⁻¹)εₜ),

∇_{σ}_{ₜ₋₁}(θₜ)=∇_{σ}_{ₜ₋₁}(Kₜ₋₁)κₜ+Kₜ₋₁∇_{σ}_{ₜ₋₁}(κₜ),

where the gradient of the matrix K, is

$${∇}_{{σ}_{t - 1}} {( K )}_{t} = [ \begin{matrix}{Q}_{t} & - {∇}_{{σ}_{t - 1}} ( {θ}_{t} ) \\ - {∇}_{{σ}_{t - 1}} {( {θ}_{t} )}^{T} & 0\end{matrix} ] {ψ}_{t}^{- 1} + {∇}_{{σ}_{t - 1}} ( {ψ}_{t}^{- 1} ) [ \begin{matrix}{K}_{t - 1} {ψ}_{t} + {θ}_{t} {θ}_{t}^{T} & - {θ}_{t} \\ - {θ}_{t}^{T} & 1\end{matrix} ] ,$$

and

Qₜ=∇_{σ}_{ₜ₋₁}(Kₜ₋₁)Ψₜ+Kₜ₋₁∇_{σ}_{ₜ₋₁}(Ψₜ)

+∇_{σ}_{ₜ₋₁}(θₜ)θₜ^{T}+θₜ∇_{σ}_{ₜ₋₁}(θₜ)^{T},

using k(xₜ, xₜ)=φₜ^{T}φₜ into Ψₜ⁻¹, then the gradient can be determined as

∇_{σ}_{ₜ₋₁}(Ψₜ⁻¹)=−(∇_{σ}_{ₜ₋₁}k(xₜ, xₜ)−∇_{σ}_{ₜ₋₁}(θₜ)ηₜ

−θₜ∇_{σ}_{ₜ₋₁}(ηₜ))Ψₜ⁻².

According to at least one implementation, a kernel recursive least squares algorithm using online kernel bandwidth can provide the main steps for the implementation of online kernel bandwidth. For example, using the kernel recursive least squares algorithm discussed above and replacing σ with σₜ, initialize η, μ, σ₁ for t=2,3, . . .

Compute the gradient of the Gaussian kernel vector,

∇_{σ}_{ₜ₋₁}(ηₜ)=└∇_{σ}_{ₜ₋₁}k(xₜ, x₁), . . . , ∇_{σ}_{ₜ₋₁}k(xₜ, xₜ₋₁)┘.

Compute the gradient of ∇_{σ}_{ₜ₋₁}(θₜ),

∇_{σ}_{ₜ₋₁}(θₜ)=∇_{σ}_{ₜ₋₁}(Kₜ₋₁)ηₜ+Kₜ₋₁∇_{σ}_{ₜ₋₁}(ηₜ).

Compute the gradient of the scaling Ψₜ,

∇_{σ}_{ₜ₋₁}(Ψₜ⁻¹)=−(∇_{σ}_{ₜ₋₁}k(xₜ, xₜ)−∇_{σ}_{ₜ₋₁}(θₜ)ηₜ

−θₜ∇_{σ}_{ₜ₋₁}(ηₜ))Ψₜ⁻².

Compute the auxiliary variable Qₜ,

Qₜ=∇_{σ}_{ₜ₋₁}(Kₜ₋₁)Ψₜ+Kₜ₋₁∇_{σ}_{ₜ₋₁}(Ψₜ)

+∇_{σ}_{ₜ₋₁}(θₜ)θₜ^{T}θₜ∇_{σ}_{ₜ₋₁}(θₜ)^{T}.

Compute the gradient of the Gram matrix ∇_{σ}_{ₜ₋₁}(Kₜ),

$${∇}_{{σ}_{t - 1}} {( K )}_{t} = [ \begin{matrix}{Q}_{t} & - {∇}_{{σ}_{t - 1}} ( {θ}_{t} ) \\ - {∇}_{{σ}_{t - 1}} {( {θ}_{t} )}^{T} & 0\end{matrix} ] {ψ}_{t}^{- 1} + {∇}_{{σ}_{t - 1}} ( {ψ}_{t}^{- 1} ) [ \begin{matrix}{K}_{t - 1} {ψ}_{t} + {θ}_{t} {θ}_{t}^{T} & - {θ}_{t} \\ - {θ}_{t}^{T} & 1\end{matrix} ] .$$

Compute the gradient of the residuals ∇_{σ}_{ₜ₋₁}(εₜ),

∇_{σ}_{ₜ₋₁}(εₜ)=−└∇_{σ}_{ₜ₋₁}(κₜ^{T})ωₜ₋₁+κₜ^{T}∇_{σ}_{ₜ₋₁}(ωₜ₋₁)┘.

Compute the auxiliary variable ω̅1ₜ,

ω̅1ₜ=∇_{σ}_{ₜ₋₁}(ωₜ₋₁)−└∇_{σ}_{ₜ₋₁}(θₜ)Ψₜ⁻¹εₜ

+θₜ∇_{σ}_{ₜ₋₁}(Ψₜ⁻¹)εₜ+θₜΨₜ⁻¹∇_{σ}_{ₜ₋₁}(εₜ)┘.

Compute the auxiliary variable ω̅2ₜ,

ω̅2ₜ=∇_{σ}_{ₜ₋₁}(Ψₜ⁻¹)εₜ+Ψₜ⁻¹∇_{σ}_{ₜ₋₁}(εₜ).

Compute the gradient of the expansion coefficients ∇_{σ}_{ₜ₋₁}(ωₜ),

∇_{σ}_{ₜ₋₁}(ωₜ)=[ω̅1ₜω̅2ₜ]^{T}.

Compute the gradient of the cost function,

∇_{σ}_{ₜ₋₁}(f_{σ}_{ₜ})=εₜ∇_{σ}_{ₜ₋₁}(εₜ).

Finally, the kernel bandwidth σₜ can be updated using equation 6.

According to at least one other implementation, coefficients can be computed using a different known algorithm, such as an extended affine projection algorithm. Once computed, the coefficients can be used in the black-box model that can then predict what a water quality value (e.g., chlorine level) can be at the next time step.

After the next time step elapses (e.g., 30 seconds), the water characteristic's current value (e.g., chlorine level) can be measured by the sensors associated with the recorder device. The predicted water quality value (e.g., chlorine level) generated by the prediction algorithm (e.g., first order autoregressive model using the kernel recursive least squares algorithm) can then be compared with the newly measured water quality value (e.g., chlorine level) and compared to calculate a residual value. If the predicted value and the measured value can be equivalent, the residual value can equal zero, indicating that the prediction can be correct. However, if the predicted value and measured value differ, the residual value can equal some number other than zero that can indicate that a contamination event can be present. The residual value's sign (i.e., positive or negative) and magnitude can indicate how the predicted value and measured value differ.

As an example, a sensor can read a water quality characteristic, such as chlorine. The measured chlorine value can be considered as an output to the model yₜ. Then, an input to the model x can be constructed that can be the lagged output yₜ. Having the input xₜ and the output y the function that links yₜ to xₜ can be determined. The function can be a black-box model that can be represented by an autoregressive model. The coefficients of the autoregressive model can then be determined. If the coefficients of the autoregressive model can be determined, then the inputs can be used to predict an output. The kernel recursive least square can be used to perform the prediction. If the predicted output is equal to the measured residual, the residual can be equal to zero. The tuning parameters characterized, for example, by the kernel bandwidth can then be left unaltered. Conversely, if the prediction starts deviating from the measured values, for example if the predicted chlorine value deviating from the measured chlorine value, then the residuals can be a nonzero value. Kernel bandwidth can also be adjusted according to the residual value by using equation 6. The advantage of this adjustment can be to accelerate the tracking, which in the context of monitoring allows to detect at the earliest possible stage any potential faults.

Next, at 212, the residual values can be analyzed by the process 200 to determine if the residuals can be deviating from zero. According to at least one implementation each residual value can be compared with zero. If the residual value equals zero, the predicted value can match the measured value and tuning parameters used by the algorithm (e.g., kernel recursive least squares algorithm) generating coefficients for the black-box model can remain fixed. Thus, the process 200 can return to 210 to predict the water quality values for the next time step.

However, if the process 200 determines that the residuals can be starting to deviate from zero at 212, the process can update tuning parameters at 214. According to at least one embodiment, tuning parameters (e.g., kernel bandwidth) within the prediction algorithm, and more specifically the algorithm used to generate coefficients (e.g., kernel recursive least squares algorithm) for the black-box model, can be updated based on the nonzero residual values. Tuning parameters can be updated to alter the prediction algorithm's water quality value (e.g., chlorine) prediction that produced the nonzero residual value. For example, if the predicted chlorine level in a water quality data at a time step differs from the actual measured chlorine level, a nonzero residual value can be produced. The tunable parameter within the coefficient algorithm can be altered using a known algorithm, such as a gradient descent method, based on the residual value. The rate that tunable parameters can be updated using the gradient descent method can be modulated by using a step-size parameter (i.e., higher step-size values indicating faster tunable parameter updating). However, the altered tunable parameter can only be used in subsequent predictions for water quality characteristic that resulted in a nonzero residual value (e.g., chlorine level) and not for other water quality characteristics (e.g., pH level). By adaptively updating tuning parameters, prediction accuracy can be improved and learning of steady-state water patterns can occur faster and lead to earlier contamination event detection. The process 200 can then use the updated tuning parameters for subsequent predictions of the water characteristic that produced the nonzero residual value at 210.

Then, at 216, water quality anomalies can be detected based on applying a change-point algorithm on the residual values. According to at least one implementation, a known algorithm, such as an online Bayesian change-point detection algorithm, can be applied to the residual values to detect water quality anomalies. The probability of the occurrence of a water contamination event can be computed based on the observed water quality values. According to at least one other implementation, statistical hypothesis testing can be applied to the residual values to detect water quality anomalies. In applying statistical hypothesis testing, residual values can be treated as variables and the mean and variance of the residual values in absence of water contamination events can be used as the basis for detecting water contamination events.

At 218, the process 200 can then transmit monitoring results to the reader device. According to at least one embodiment, the recorder device can only transmit monitoring results to the reader device as an alert (i.e., warning message) if a water contamination event can have been detected at 216. Additionally, the reader device can not be activated until the recorder device has sent an alert in order to reduce resources needed by the reader device. Monitoring results can include the anomalous water quality value (e.g., chlorine value), the location that the anomalous water quality value was detected, and the severity of the anomaly.

It can be appreciated that FIG. 2 provides only an illustration of one implementation and do not imply any limitations with regard to how different embodiments can be implemented. Many modifications to the depicted embodiment can be made based on design and implementation requirements.

The above embodiment provides advantages over traditional water quality monitoring methods by allowing faster contamination detection and fewer resources to be used in monitoring. In the case of a mobile recorder device (e.g., self-propelled submersible robot), the mobile recorder device can monitor many water locations without requiring the expense associated with multiple fixed sensors. Using a mobile recording device can also allow for more flexibility in changing sampling locations over fixed sensors. However, the above embodiment can be implemented with multiple fixed sensors.

Furthermore, utilizing tunable parameters in conjunction with a prediction algorithm can provide more accurate predictions of water quality values and rapid initialization to enable early contamination event detection. Less time can be needed to implement corrective actions in response to contamination events since the exact location, time, and associated variables can be known in advance. Resources for monitoring water quality can also be reduced as the reader device can only receive data from the recorder device once a contamination event can be detected, thus the reader device can not need to be continuously activated.

FIG. 3 is a block diagram 300 of internal and external components of computers depicted in FIG. 1 in accordance with an illustrative embodiment of the present invention. It should be appreciated that FIG. 3 provides only an illustration of one implementation and does not imply any limitations with regard to the environments in which different embodiments can be implemented. Many modifications to the depicted environments can be made based on design and implementation requirements.

Data processing system 800, 900 is representative of any electronic device capable of executing machine-readable program instructions. Data processing system 800, 900 can be representative of a smart phone, a computer system, PDA, or other electronic devices. Examples of computing systems, environments, and/or configurations that can represented by data processing system 800, 900 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 (FIG. 1), and network server 110 (FIG. 1) can include respective sets of internal components 800 a, b and external components 900 a, b illustrated in FIG. 3. Each of the sets of internal components 800 a, b includes one or more processors 820, one or more computer-readable RAMs 822 and one or more computer-readable ROMs 824 on one or more buses 826, and one or more operating systems 828 and one or more computer-readable tangible storage devices 830. The one or more operating systems 828 and programs such as a water quality monitoring program 108a and 108b (FIG. 1), can be stored on one or more computer-readable tangible storage devices 830 for execution by one or more processors 820 via one or more RAMs 822 (which typically include cache memory). In the embodiment illustrated in FIG. 3, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory or any other computer-readable tangible storage device that can store a computer program and digital information.

Each set of internal components 800 a, b also includes a R/W drive or interface 832 to read from and write to one or more portable computer-readable tangible storage devices 936 such as a CD-ROM, DVD, memory stick, magnetic tape, magnetic disk, optical disk or semiconductor storage device. The water quality monitoring program 108a and 108b (FIG. 1) can be stored on one or more of the respective portable computer-readable tangible storage devices 936, read via the respective R/W drive or interface 832 and loaded into the respective hard drive 830.

Each set of internal components 800 a, b can also include network adapters (or switch port cards) or interfaces 836 such as a TCP/IP adapter cards, wireless wi-fi interface cards, or 3G or 4G wireless interface cards or other wired or wireless communication links. The water quality monitoring program 108a (FIG. 1) in client computer 102 (FIG. 1) and the water quality monitoring program 108b (FIG. 1) in network server computer 110 (FIG. 1) can be downloaded from an external computer (e.g., server) via a network (for example, the Internet, a local area network or other, wide area network) and respective network adapters or interfaces 836. From the network adapters (or switch port adaptors) or interfaces 836, the water quality monitoring program 108a (FIG. 1) in client computer 102 (FIG. 1) and the water quality monitoring program 108b (FIG. 1) in network server computer 110 (FIG. 1) are loaded into the respective hard drive 830. The network can comprise copper wires, optical fibers, wireless transmission, routers, firewalls, switches, gateway computers and/or edge servers.

Each of the sets of external components 900 a, b can include a computer display monitor 920, a keyboard 930, and a computer mouse 934. External components 900 a, b can also include touch screens, virtual keyboards, touch pads, pointing devices, and other human interface devices. Each of the sets of internal components 800 a, b also includes device drivers 840 to interface to computer display monitor 920, keyboard 930 and computer mouse 934. The device drivers 840, R/W drive or interface 832 and network adapter or interface 836 comprise hardware and software (stored in storage device 830 and/or ROM 824).

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for water quality monitoring and early detection of a contamination event by a recorder device, the method comprising:
receiving a first water characteristic value corresponding with a water characteristic;
determining a second water characteristic value corresponding with the water characteristic using a prediction algorithm having a tunable parameter, based on the first water characteristic value and the tunable parameter;
comparing the first water characteristic value and the second water characteristic value;
determining a residual value, based on comparing the first water characteristic value to the second water characteristic value;
updating the tunable parameter, based on the determined residual value;
determining if the contamination event is present, based on the residual value; and
sending an alert, based on determining the presence of the contamination event.

2. The method of claim 1, wherein the first water characteristic value has a location value and a time value.

3. The method of claim 2, wherein the alert comprises the location value, the time value, and the water quality characteristic.

4. The method of claim 1, wherein the prediction algorithm comprises at least one of a kernel recursive least squares algorithm or an extended affine projection algorithm.

5. The method of claim 1, wherein the recorder device comprises at least one of a fixed sampling station or a mobile sampling station.

6. The method of claim 1, wherein the residual value comprises a sign and a magnitude.

7. The method of claim 6, wherein the updating the tunable parameter comprises updating the tunable parameter based on the sign and the magnitude of the residual value.

8. A computer system for water quality monitoring and early detection of a contamination event by a recorder device, comprising:
a processor, a computer-readable memory, a computer-readable tangible storage medium, and program instructions stored on the tangible storage medium for execution by at least one of the one or more processors via at least one of the one or more memories, wherein the computer system is capable of performing a method comprising:
receiving a first water characteristic value corresponding with a water characteristic;
determining a second water characteristic value corresponding with the water characteristic using a prediction algorithm having a tunable parameter, based on the first water characteristic value and the tunable parameter;
comparing the first water characteristic value and the second water characteristic value;
determining a residual value, based on comparing the first water characteristic value to the second water characteristic value;
updating the tunable parameter, based on the determined residual value;
determining if the contamination event is present, based on the residual value; and
sending an alert, based on determining the presence of the contamination event.

9. The computer system of claim 8, wherein the first water characteristic value has a location value and a time value.

10. The computer system of claim 9, wherein the alert comprises the location value, the time value, and the water quality characteristic.

11. The computer system of claim 8, wherein the prediction algorithm comprises at least one of a kernel recursive least squares algorithm or an extended affine projection algorithm.

12. The computer system of claim 8, wherein the recorder device comprises at least one of a fixed sampling station or a mobile sampling station.

13. The computer system of claim 8, wherein the residual value comprises a sign and a magnitude.

14. The computer system of claim 13, wherein the updating the tunable parameter comprises updating the tunable parameter based on the sign and the magnitude of the residual value.

15. A computer program product for water quality monitoring and early detection of a contamination event by a recorder device, comprising:
a computer-readable storage medium and program instructions stored on the tangible storage medium, the program instructions executable by a processor, the program instructions comprising:
program instructions to receive a first water characteristic value corresponding with a water characteristic;
program instructions to determine a second water characteristic value corresponding with the water characteristic using a prediction algorithm having a tunable parameter, based on the first water characteristic value and the tunable parameter;
program instructions to compare the first water characteristic value and the second water characteristic value;
program instructions to determine a residual value, based on comparing the first water characteristic value to the second water characteristic value;
program instructions to update the tunable parameter, based on the determined residual value;
program instructions to determine if the contamination event is present, based on the residual value; and
program instructions to send an alert, based on determining the presence of the contamination event.

16. The computer program product of claim 15, wherein the first water characteristic value has a location value and a time value.

17. The computer program product of claim 16, wherein the alert comprises the location value, the time value, and the water quality characteristic.

18. The computer program product of claim 15, wherein the prediction algorithm comprises at least one of a kernel recursive least squares algorithm or an extended affine projection algorithm.

19. The computer program product of claim 15, wherein the program instructions to send the alert comprises sending the alert to a reader device.

20. The computer program product of claim 15, wherein the recorder device comprises at least one of a fixed sampling station or a mobile sampling station.
